SEQUENCE LISTING (6 sequences)

SEQ ID NO: 1 - DNA, 12 bp, Artificial Sequence
  Chemically Synthesized, Synthetic Construct
tttttttttttt

SEQ ID NO: 2 - DNA, 20 bp, Artificial Sequence
  Chemically Synthesized, Synthetic Construct
agccttgtacttcgtatggc

SEQ ID NO: 3 - DNA, 12 bp, Artificial Sequence
  Chemically Synthesized, Synthetic Construct
aaaaaaaaaaaa

SEQ ID NO: 4 - DNA, 20 bp, Artificial Sequence
  Chemically Synthesized, Synthetic Construct
tcggaacatgaagcataccg

SEQ ID NO: 5 - DNA, 9 bp, Artificial Sequence
  Chemically Synthesized, Synthetic Construct
tagtagtag

SEQ ID NO: 6 - DNA, 8 bp, Artificial Sequence
  Chemically Synthesized, Synthetic Construct
tttatttt

(19) United States
(12) Patent Application Publication
(10) Pub. No.: US 2011/0003703 A1
(43) Pub. Date: Jan. 6, 2011

(54) Nucleic Acid Hybridization and Detection Using Enzymatic Reactions on a Microarray

(76) Inventors: Charles Ma, Palo Alto, CA (US); Yulin Lee, Hsinchu (TW)

Correspondence Address: DERGOSITS & NOAH LLP, Three Embarcadero Center, Suite 410, SAN FRANCISCO, CA, 94111, US

(21) Appl. No.: 12/496,613

(22) Filed: Jul. 1, 2009

(51) Int. Cl. C40B 30/04; C12Q 1/68; C40B 60/12

(52) U.S. Cl. 506/9; 435/6; 506/39

(57) ABSTRACT

Embodiments are directed to a methods and systems for nucleic acid detection using enzymatic reactions on a microarray. In one embodiment, a probe comprising a probe nucleotide sequence and a substantially homogenous sequence extender portion is provided on the surface of a microarray. The probe nucleotide sequence is hybridized to the complementary target nucleotide sequence. A solution containing enzymes and detection elements is applied to the hybridized probe structure. The enzyme determines the composition of the nucleotide structure of the extender and creates a complementary homogenous sequence extender structure between the target nucleotide sequence and the microarray surface structure. The detection elements in the solution are bound to the extender structure, thus allowing detection using an appropriate detector system.

### TECHNICAL FIELD

Embodiments relate generally to methods and systems for analysis of nucleic acids, and more specifically to detection and measurement of RNA using biological microarrays.

### BACKGROUND

Gene expression analysis typically relies on the detection and characterization of nucleic acid sequences and variations in nucleic acid sequences in a sample. Various methods have been developed to detect and characterize specific nucleic acid-sequences and sequence variants. With the completion of the nucleic acid sequencing of the human genome, as well as the genomes of numerous pathogenic organisms, the need for efficient and cost-effective tests for the detection of specific nucleic acid sequences continues to grow. In general, these tests must be able to create a detectable signal from samples that contain very few copies of the sequence of interest.

The development of microarrays (also referred to as “biochips”) has greatly advanced RNA detection and analysis processes. A microarray is a substrate, such as a glass slide, a silicon wafer, metal slide, a nylon film or other polymer-based substrate, that contains a plurality of different reagents immobilized on the surface. These reagents (known as “probes”) are selected for their high specificity in binding affinity or reactivity toward their counterparts (known as “targets”) in biological samples. The probes are composed of nucleic acids with a complementary sequence to all or part of the RNA of interest, and can be DNA, RNA, or oligonucleotides with a minimum of 6 to 8 (and more commonly 19-24) complementary bases to the target sequence. After applying a biological sample onto a microarray under an experimentally controlled condition, the interactions between each probe on a microarray and its corresponding target in the biological sample can be observed through various target labeling techniques and appropriate detection instrumentation, thus providing the microarray user with qualitative and quantitative information about the tested biological sample.

The total RNA of a sample comprises the purified RNA from tissue, and contains all the RNA of the cells. The general types of RNA include large non-coding RNA, small non-coding RNA (e.g., snRNA, miRNA, tRNA, and so on), Ribosomal RNA and messenger RNA (mRNA). Short non-coding RNA, such as microRNA (miRNA) are potent regulators of gene expression. In genetics, miRNAs are single-stranded RNA molecules of about 19-23 nucleotides (nt) in length. A small number of miRNAs have been identified, due in part to the practical challenges associated with present detection methods. In general, miRNAs hybridize to mRNAs with one or more mismatches. Furthermore, miRNAs are generally too short for conventional DNA probes to be effective. These challenges often cause too many non-specific signals during the detection process.

Present techniques for detecting miRNAs include enrichment techniques that amount to a size selection process that operates to isolate RNA molecules smaller than a specific size (e.g., 200 nt). These usually include miRNA, snRNA (small nuclear RNA), snoRNA (small nucleolar RNA), small antisense/non-coding RNA (bacterial), small ribosomal RNA and tRNA. Drawbacks to this technique include a relatively low yield (for example, the fraction of miRNAs in the total RNA pool may be less than 0.1%), an increase in variability, and the amount of work required to perform the enrichment. The enrichment process also requires a large amount of starting material, for example on the order of 5-10 micrograms in a typical experimental procedure.

Another method that has been developed to overcome the challenge of using total RNA for miRNA hybridization is the incorporation of Locked Nucleic Acid (LNA) into the probe. An LNA is a modified RNA nucleotide in which the affinity for target is increased resulting in more stable duplexes that provide sensitivity and specificity to detect tissue-specific RNAs. This technique, however, requires the use of special processing steps and can be a relatively expensive and involved process.

Another approach is the use of the small hairpin RNA, which is a sequence of RNA that makes a tight hairpin-shaped turn that can be used to silence gene expression through RNA interference. The hairpin RNA helps stabilize specific interaction and destabilizes non-specific interactions. Like the LNA approach, this technique also requires special processing steps and can implicate expensive and proprietary processes.

Present diffusion-based hybridization methods typically require a tradeoff between the specificity of the detection and the sensitivity of detection. In general, an increase in sensitivity requires a reduction in specificity, and vice-versa. Thus, typical methods and systems that make it easy to detect bound RNA sequences (high sensitivity) may make it difficult to identify the detected sequences (low specificity). Conversely, systems that are optimized to identify and distinguish sequences generally suffer from low sensitivity, in that a relatively low number of target sequences are bound.

Other disadvantages associated with present hybridization methods include the need to purify the amount of source material to remove large RNA compounds in order to decrease background signals during the detection processes, the requirement for a large amount of source material (e.g., 5 μg or greater of total RNA), or the need to use degraded RNA, which can cause the occurrence of non-specific signals and compromise detection results. Another disadvantage associated with certain known hybridization techniques include strict and limited temperature ranges for hybridization (e.g., 37-42° C. for 23mer oligonucleotides) and inconsistent results at different hybridization temperatures an/or loss of weak signals at higher temperature ranges. Yet another disadvantage associated with common hybridization methods includes complex processing steps, such as the requirement of at least two separate low-stringency wash cycles, and long hybridization periods (e.g., 8-20 hours).

What is desired, therefore, is a nucleic acid hybridization and detection system that binds a high number of target nucleotide sequences without sacrificing the specificity of identification. What is further desired is a hybridization and detection system that does not require a relatively large amount of starting material and that allows for hybridization to occur in a short period of time and under a wide range of operational conditions.

### SUMMARY OF THE INVENTION

Embodiments are directed to methods and systems for nucleic acid (e.g., miRNA) hybridization and detection using enzymatic reactions on a microarray. In one embodiment, a probe comprising a probe nucleotide sequence and a homogenous sequence extender portion is provided on the surface of a microarray. The probe nucleotide sequence is hybridized to the complementary target nucleotide sequence. A solution containing enzymes and detection elements is applied to the hybridized probe structure. The enzyme reacts upon the nucleotide structure of the extender and creates a complementary extender structure of a complementary homogenous sequence between the target nucleotide sequence and the microarray surface structure. The detection elements in the solution are bound to the extender structure, thus allowing detection using an appropriate detector system. In an alternative embodiment, a single solution containing the target nucleotide, enzyme, and detection elements is applied to the probe in a single application step. The hybridization of the probe and target nucleotide sequences occurs concurrently with the creation of the complementary extender structure and the binding of the detection elements to the extender. In this embodiment, all of the hybridization process effectively ends when all of the complementary target sequences in the solution are bound to the probe.

### BRIEF DESCRIPTION OF THE DRAWINGS

Embodiments are illustrated by way of example and not limitation in the figures of the accompanying drawings, in which like references indicate similar elements and in which:

FIG. 1A illustrates a portion of a microarray biochip on which an enzymatic reaction is initiated to bind detection elements to a probe through the formation of a complementary extender structure, under an embodiment.

FIG. 1B illustrates a portion of a microarray biochip on which an enzymatic reaction has progressed to a first stage to form a complementary extender structure, under an embodiment.

FIG. 1C illustrates a portion of a microarray biochip on which an enzymatic reaction has progressed to a final stage to form a complementary extender structure, under an embodiment.

FIG. 1D illustrates the portion of the microarray biochip of FIG. 1C with example nucleotide sequences provided including a homogenous sequence extender portion.

FIG. 2 is a flowchart illustrating a method of performing a nucleic acid hybridization process using enzymatic reactions, under an embodiment.

FIG. 3 illustrates an example detectable hybridized probe-target structure after hybridization and enzymatic reaction, under an embodiment.

FIG. 4 is a flowchart that illustrates a concurrent hybridization and enzymatic reaction process for producing a detectable hybridized probe sequence, under an embodiment.

FIG. 5 illustrates a nucleic acid detection system for use with a microarray hybridized through an enzymatic reaction, under an embodiment.

### INCORPORATION BY REFERENCE

Each publication and/or patent mentioned in this specification is herein incorporated by reference in its entirety to the same extent as if each individual publication or patent was specifically and individually indicated to be incorporated by reference.

### DETAILED DESCRIPTION

Embodiments of a microarray biochip comprising a substrate, a plurality of reagents (probes) immobilized on the substrate, are described. A probe comprising a probe nucleotide sequence and an extender portion is provided on the surface of a microarray. The probe nucleotide sequence is hybridized to the complementary target nucleotide sequence. A solution containing enzymes and detection elements is applied to the hybridized probe structure. The enzyme reacts upon the nucleotide structure of the extender and creates a complementary extender structure between the target nucleotide sequence and the microarray surface structure. The detection elements in the solution are bound to the newly-formed complementary extender structure, thus allowing detection using an appropriate detector system.

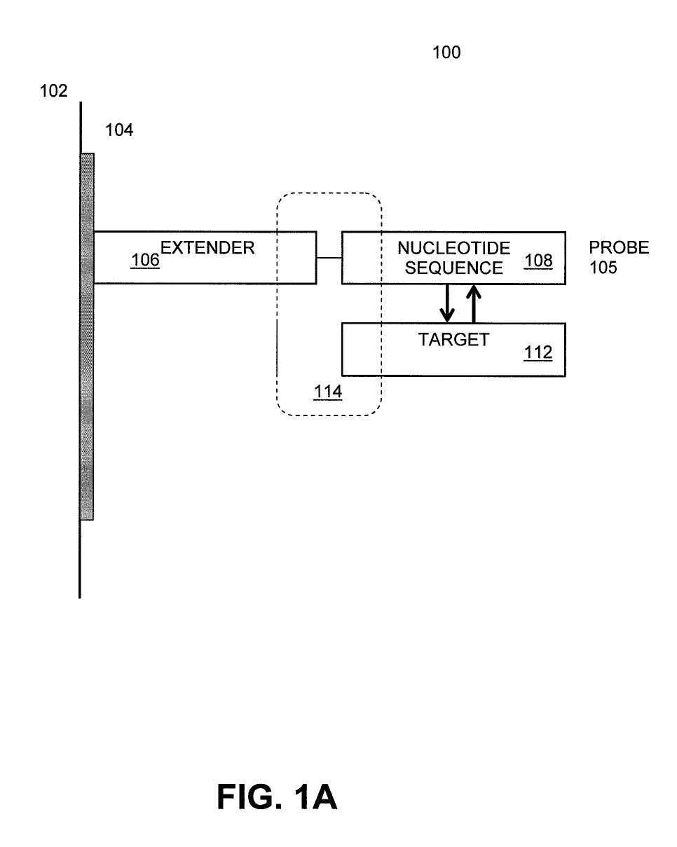

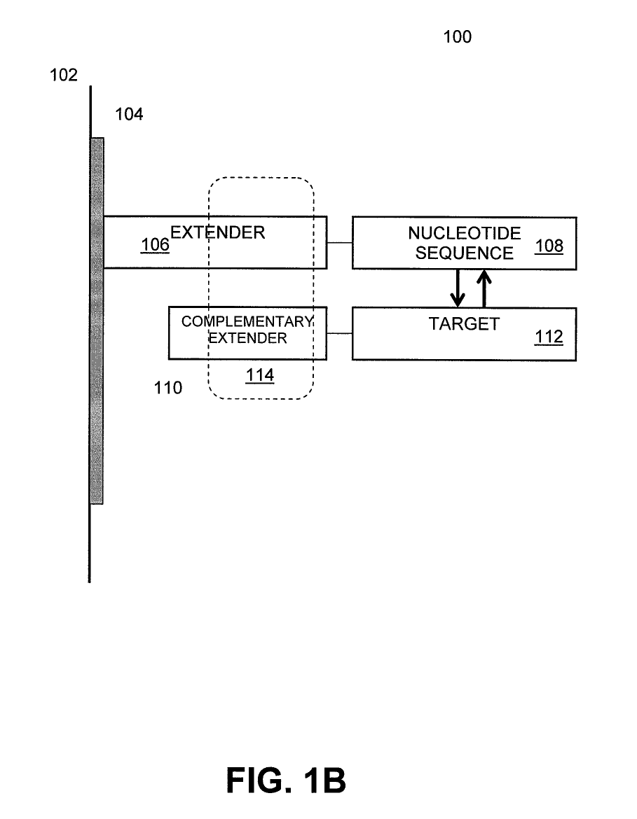

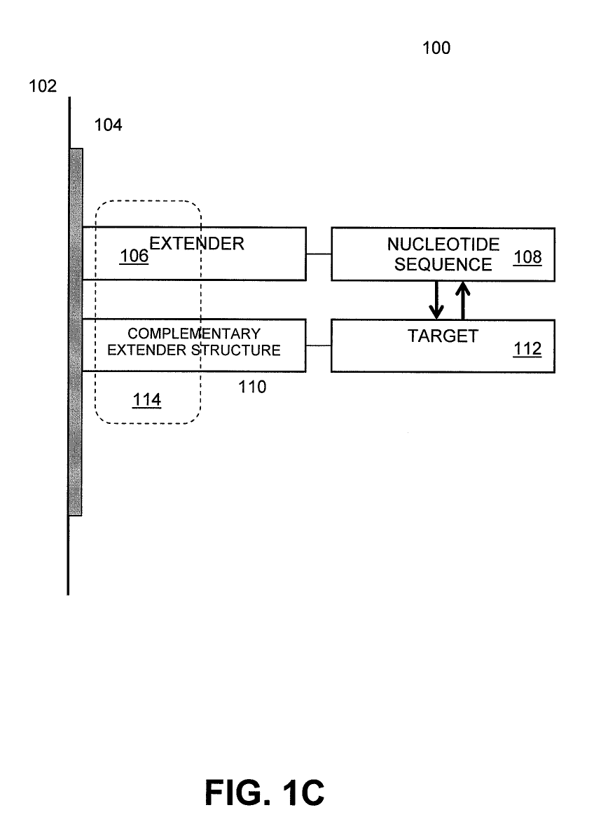

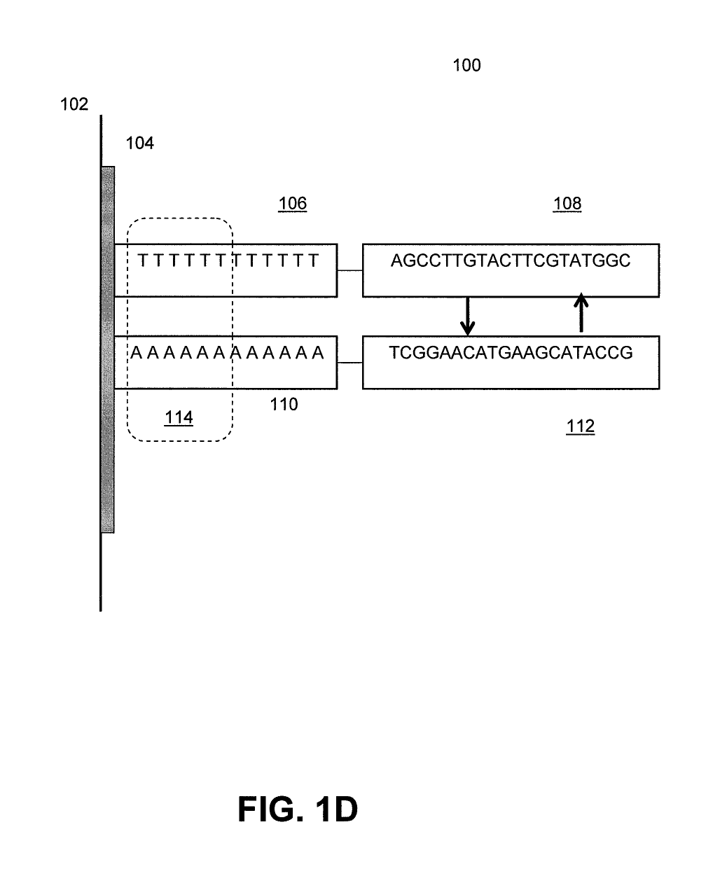

FIG. 1A illustrates a portion of a microarray biochip on which an enzymatic reaction is initiated to bind detection elements to a probe through the formation of a complementary extender structure, under an embodiment. The microarray biochip 100 comprises a slide, or similar type of substrate 102 and a probe region 105. The substrate 102 can be made of any suitable material that allows nucleic acids to be immobilized, such as glass, plastic, fused silica, silicon, ceramic, metal, cellulose membranes, epoxide-based porous surfaces, and the like. The substrate may be formed in any appropriate shape, such as a rectangle, square, circle, triangle, polygon, or any other convenient and substantially planar shape. On a microarray, the probe region 105 represents one of many probes (reagents) deposited on the substrate 102 in an arrayed manner. Depending on the type of reagents used in the probe 105, a microarray biochip 100 can be embodied in biochips such as a gene biochip, DNA biochip, oligonucleotide microarray biochip, polynucleotide microarray biochip, protein microarray biochip, antibody microarray biochip, and any other similar type of biochip.

As shown in FIG. 1A, the probe 105 comprises a nucleotide sequence that is selected for a high specificity in binding affinity or reactivity toward a counterpart or complement in biological samples. As used herein, the term “probe” refers to an oligonucleotide or nucleic acid that hybridizes to a target sequence to facilitate detection of the target sequence. Hybridization refers to the chemical reaction between the probe and the target DNA or RNA to be detected, and occurs through the binding or annealing of complementary or matched nucleic acid sequences of the probe and target. Complementary sequences may be sequences that have at least 50% sequence identity, although it is generally preferred to have 100% sequence identity to constitute a hybridized sequence. As used herein, the term “nucleotide” refers to any of various compounds consisting of a nucleoside combined with a phosphate group and forming the basic constituent of DNA and RNA, and can include synthetic nucleotide analogs that can be the subject of enzymatic action.

After applying a biological sample onto the substrate 102 with the reagents in probe region 105 under experimentally controlled conditions, the interactions between each reagent on the substrate 102 and its corresponding target in the biological sample can be observed through various target labeling techniques and appropriate detection instrumentation, thus providing the microarray user with qualitative and quantitative information about the target in the tested biological sample.

In one embodiment, the probe nucleotide sequence 108 can be virtually any compound that binds to a target with a sufficient specificity, such as nucleic acids that bind to complementary nucleic acid targets through Watson-Crick and/or Hoogsteen binding. The probe nucleotide sequence 108 can by any specific sequence of nucleic acid elements, such as DNA, RNA, PNA and LNA elements in a linear arrangement of contiguous nucleotides. The length and composition of the nucleotide sequence depends on the application and nature of the target. A typical length may be on the order of 5-25 nucleotides, but lengths can range from as little as four nucleotides to over 1000 nucleotides or more, depending on the application.

For the example of FIG. 1A, the probe 105 also includes an extender portion 106 that separates the nucleotide sequence 108 from the substrate surface 102. The extender 106 provides a degree of spatial separation of the nucleotide sequence 108 from the substrate surface, and provides a structure for binding of the detection elements. In an embodiment, the extender is non-reactive with respect to hybridization, but is configured to allow an enzymatic reaction to perform certain processes. The extender 106 may be composed of any appropriate sequence of nucleotide elements and of a certain length depending upon the application and nature of the target. For example, the extender may be composed of a number of nucleotide elements in a sequence structure that is 10-30 nucleotides in length. The length may be the same or similar to the length of the actual probe nucleotide sequence 108. The extender may be a homogeneous or mono sequence of a number of single nucleotide elements, such as a poly-T or poly-A structure, or it may be a repeating sequence of two or more nucleotide elements, such as TAGTAGTAG. Alternatively, the extender can be any recognizable sequence of nucleotides that is different from the probe nucleotide sequence, and may even be a random sequence, such as TTTATTTT, or the like. In general, a homogeneous (single element) structure may be preferred, since the use of two or more nucleotide sequences can cause random extension problems, such as nonspecific false positives.

In an embodiment, the extender may include one or more linker or spacer structures that serve to separate the probe nucleotide sequence from the surface of the substrate. The spacer can be any of a variety of non-active or inert molecules, such as nucleotides, phospholipids, amino acids, alkyl and alkenyl carbonates, and the like. Essentially, any molecule having the appropriate size characteristics and capable of being linked to the probe and any detection elements can be used as a linker or spacer.

In a typical application, a solution containing a target of interest is applied to the microarray. As used herein, a “target solution” or “sample” refers to any liquid or semi-liquid composition that contains a target nucleic acid or extracted nucleic acid to be analyzed. The target solution may be a biological sample, such as any type of biological fluid. With reference to FIG. 1A, upon applying the target solution to the probe 105, the probe nucleotide sequence 108 hybridizes to the appropriate target sequence 112. This entails the probe sequence binding to the target sequence to form a hybrid or hybridized nucleotide sequence.

A label or reporter molecule is used to report the site of the hybridization of the probe. A label generally refers to any chemical group or moiety having a detectable physical property or any compound capable of causing a chemical group or moiety to exhibit a detectable physical property, or inhibit the expression of a particular physical property. The physical properties may include visual, electrical, radioactive, biological, or other discernible properties that can be detected through an appropriate detection system. In an embodiment, the label or reporter is implemented through the linkage of detection elements to the hybridized probe area or an area adjacent to or associated with the hybridized probe area. As used herein, the term “detection element” refers to a portion of the label or reporter that is detectable. With respect to the enzymatic reaction that is performed on the substrate, the term “detection element” includes the nucleotide triphospate piece that is the subject of enzymatic action and the piece that is detectable; in the case of radioactives, one element of the nucleotide is a detectable isotope, and in the case of visuals or biologicals, dyes or biotin are conjugated to the nucleotide triphosphate molecule to produce detectable elements.

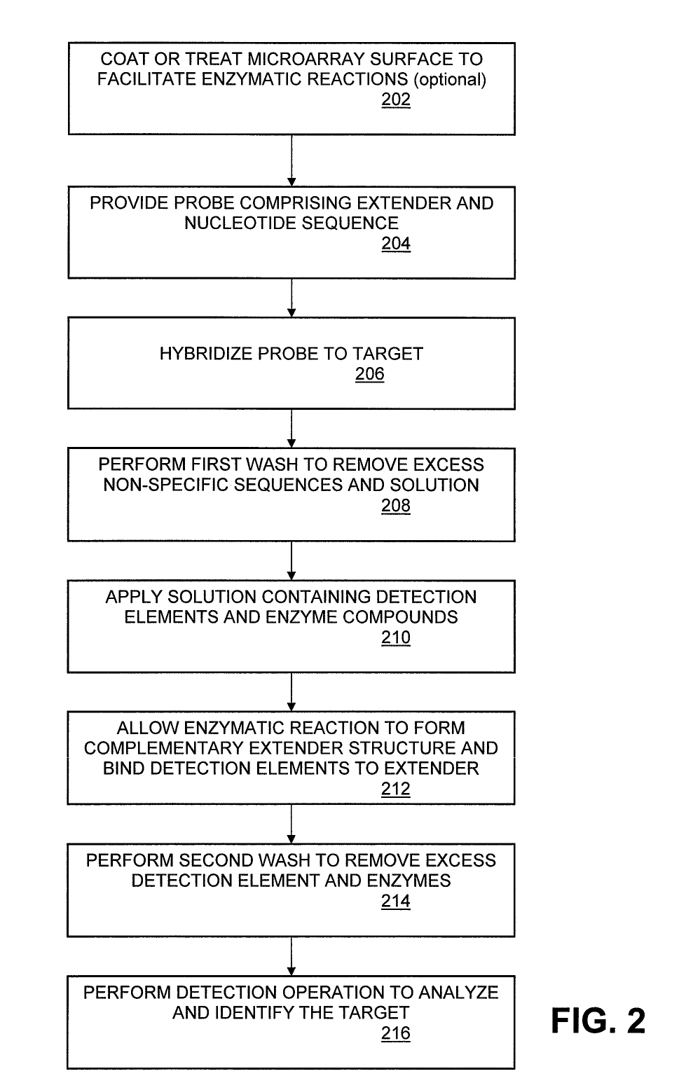

In an embodiment, an enzymatic solution is used to bind detection elements to the probe 105 to facilitate detection and analysis of the target 112. FIG. 2 is a flowchart illustrating a method of performing a nucleic acid hybridization process using enzymatic reactions, under an embodiment. As shown in FIG. 2, the process starts by providing a probe comprising an extender 106 and nucleotide sequence, block 204. The process may include an optional surface treatment step 202 involving coating or treating the substrate surface 102 to facilitate enzymatic reactions. Thus, in one embodiment, the microarray 100 of FIG. 1A may include a protein coating 104 or other similar coating or surface treatment that encourages enzymatic reactions on the surface of the substrate 102. Such a coating may not be required in certain applications, such as when a sufficient amount of reaction material is provided for the hybridization process and/or if speed of the reaction is not critical. The protein coating acts to stabilize the protein structure near the surface of the substrate, and is configured to help overcome any problems associated barriers or impediments to an enzymatic reaction, such as through hydrophobicity, electrostatic effects, steric hindrance, and the like. In an alternative embodiment, instead of an actual separate coating layer, the substrate surface 102 may be treated through a physical or chemical process to facilitate or accelerate enzymatic reactions on the surface. In an embodiment, certain reagents, such as bovine serum albumin (BSA) can be added to the substrate surface to increase the stability of protein structures to facilitate the enzymatic reaction.

In block 206 of FIG. 2, the target solution is introduced to enable hybridization of the target sequence 112 to the probe sequence 108. After hybridization, a first wash operation is then performed to remove any excess solution compounds, such as non-specific nucleic acid sequences and other random or unwanted compounds. The hybridization step may be performed under conditions appropriate to the particular application, and under controlled environmental conditions, such as ambient or applied temperature conditions, and the like. The stringency of hybridization may range from low to medium to high stringency, as required, and may be achieved using specific buffers, salts, and/or temperatures. Typical hybridization periods may be between 8 to 20 hours, depending upon the application, and the end of the hybridization process may be marked by the expiration of a set period of time, a defined change in the target solution, or any other defined terminating condition.

After the hybridization step, as shown in block 210 of FIG. 2, a buffer solution containing enzymes and detection elements is provided to the hybridized target. The enzyme in the enzymatic solution binds onto the area between the probe nucleotide sequence 108 and the extender 106. With reference to FIG. 1A, the area 114 represents the initial enzymatic reaction area. The enzyme binds to the binding area through an enzymatic kinetic reaction (i.e., an electrostatic, hydrophobic, or protein-nucleic acid reaction). As the enzymatic reaction progresses, it moves along the extender structure 106 to form a complementary extender structure 110 that is bound to the target 112, as shown in block 212 of FIG. 2. FIG. 1B illustrates a portion of a microarray biochip on which an enzymatic reaction has progressed to a first stage to form a complementary extender structure, under an embodiment. As the enzymatic reaction progresses, the reaction area 114 moves down the extender 106. The enzyme acts upon the composition of the extender structure (e.g., a poly-A or poly-T sequence) and automatically selects the complementary nucleotides from the compounds in the enzymatic solution (e.g., either dTTP or dATP). Thus, if the extender 106 is a poly-T structure, the enzymatic reaction pulls dATP from the solution and forms a poly-A structure 110; likewise, if the extender 106 is a poly-A structure, the enzymatic reaction pulls dTTP from the solution and forms a poly-T structure 110. The enzymes thus automatically recognize the composition of the extender 106 (e.g., A or T molecules) and select the complementary compounds from the solution (e.g., T or A molecules) and then create the complementary structure 110 which is bound to the target 112. In this manner, the enzyme selectively permits reaction with the proper nucleotides. The embodiment of FIG. 1B illustrates a case in which the enzymatic reaction has progressed to a point where a portion of the complementary extender 110 is formed. FIG. 1C illustrates a portion of a microarray biochip on which an enzymatic reaction has progressed to a final stage to form a complementary extender structure, under an embodiment. As shown in FIG. 1C, the enzymatic reaction may finish before the end of the extender 106 is reached and may not reach the surface of the slide due to physical constraints associated with the molecular characteristics and other factors.

FIG. 1D illustrates the portion of the microarray biochip of FIG. 1C with example nucleotide sequences provided including a homogenous sequence extender portion. As shown in FIG. 1D, the extender 106 is a homogenous poly-T sequence. As the enzymatic reaction 114 progresses, a complementary poly-A sequence 110 is created.

In one embodiment, the enzymatic solution used for the enzymatic reaction 114 includes detection elements in the form of conjugated dATP or dTTP molecules. The detection elements can comprise any appropriate labeling technology, such as fluorescent molecules (e.g., fluorescein, rhodamine, Cy-3, Cy-5, and so on), a dye, a chemiluminescent molecule, a bioluminescent molecule, a radioisotope (e.g., P³² or H³, ¹⁴C, ¹²⁵I, and ¹³¹I), an electrical charge transducing molecule, and electromagnetic molecule, a nuclear magnetic resonance molecule, and the like. The detection element can also be provided in the form of an indirectly detectable (biological) label such, as an enzyme, a hapten (e.g., biotin, pyridoxal, etc), an antibody, and the like. The detection elements are effectively bound to the extender structure 106 through the complementary extender 110. This then provides the portion of the probe that can be detected through the appropriate detection apparatus.

The enzymatic solution may include any appropriate enzyme that catalyzes the chemical reactions on the surface of the substrate 102. In one embodiment, the enzymatic solution contains Klenow, which is a large protein fragment produced when DNA polymerase I is enzymatically cleaved by the protease subtilisin, and lacks the 5′→3′ polymerase activity. The enzyme can also be the exo-Klenow fragment, which lacks any exonuclease activity (5′→3′ or 3′→5′). Besides Klenow, any other appropriate enzyme, such as BLST1, VENT polymerase, or Therminator II DNA polymerase may be used.

For an embodiment in which the enzymatic solution contains exo-Klenow, the step 212 of allowing the enzymatic reaction to bind the detection elements to the extender comprises incubating the hybridized probe with the Klenow and a biotin-dATP or biotin-dTTP solution for a defined period of time (e.g., 1 hour) and at a defined temperature (e.g., 37 degrees C.). The Klenow is then bound with an avidin-Cy5 solution, or any other appropriate detection element and avidin. This effectively binds the detection element to the extender region of the probe.

With reference to FIG. 2, in block 214 of FIG. 2, a second wash is performed to remove the excess enzyme solution and detection elements, and any other non-required compounds. At this point, the microarray is ready to be used in a detection operation to analyze and identify the target, block 216.

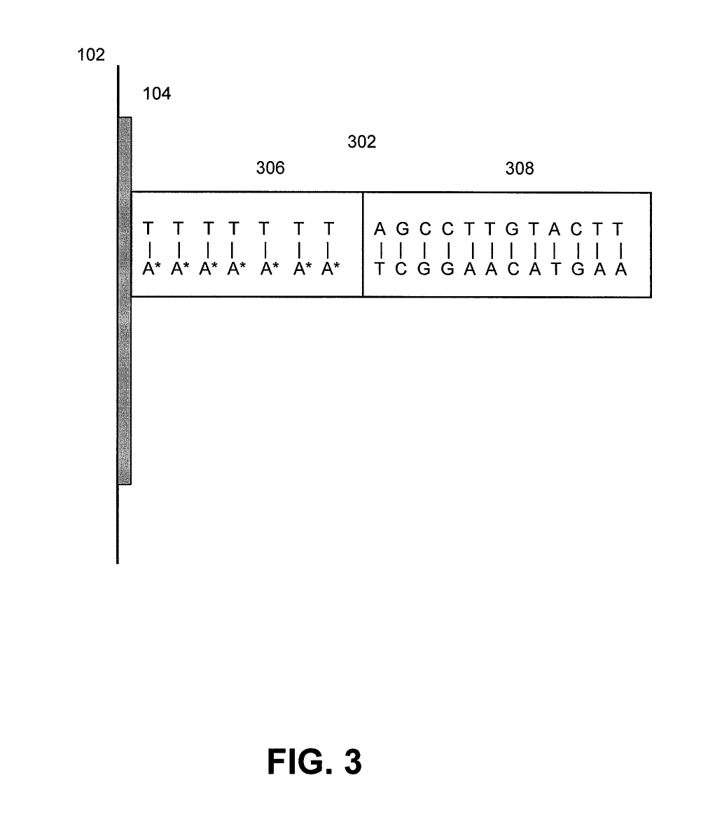

The process of FIG. 2 results in the creation of a hybridized probe sequence that has detection elements bound to an extender structure to allow detection of the bound target. The use of the enzymatic reaction allows the binding of detection elements in a manner that greatly reduces the amount of target solution material required. This process increases the sensitivity and selectivity of the hybridization process. FIG. 3 illustrates an example detectable hybridized probe-target structure after hybridization and enzymatic reaction, under an embodiment. As shown in FIG. 3, the hybridized probe nucleotide sequence 308 is bound to the extender section 306 that includes the detection elements. The example sequences of FIG. 3 illustrate the poly-T sequence of the original extender bound with the poly-A of the complementary extender structure formed by the enzymatic reaction. The T-A bond for the extender is a standard base-pair hydrogen bond. The complementary extender structure is denoted A* to indicate that the poly-A structure is covalently conjugated with an appropriate marker, such as a covalently conjugated dye or covalently conjugated biotin. The detection elements are thus bound to the extender 306 to allow detection of the hybridized probe sequence 308 through perception of the electrical, visual, radioactive, or other signals emitted by the detection elements, through an appropriate detection system. The enzymes 114 that were used to build the complementary extender structure 110 either dissipated from the hybridized probe region naturally and/or were washed off during the second wash step, 214.

The embodiment of FIG. 2 illustrated a method in which two solutions, a target solution and a separate enzyme solution were provided to perform two separate reactions, a hybridization reaction and an enzymatic reaction on the substrate surface. In an embodiment, a single solution may be provided so that the hybridization reaction occurs concurrently with the enzymatic reaction on the substrate surface.

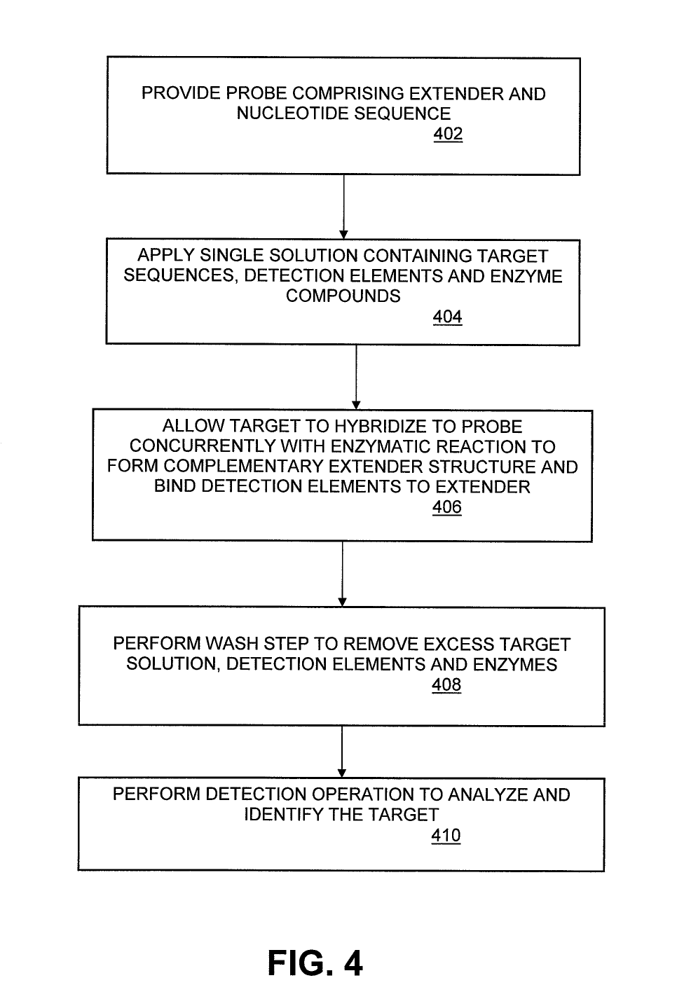

FIG. 4 is a flowchart that illustrates a concurrent hybridization and enzymatic reaction process for producing a detectable hybridized probe sequence, under an embodiment. As shown in FIG. 4, a probe comprising a spacer and probe nucleotide sequence is provided, block 402. To this probe is applied a single solution containing the target sequences, the enzyme solution, and the detection elements, block 404. Application of this single solution allows the hybridization step of the probe and target to occur concurrently with the enzymatic reaction that creates the complementary extender structure and binding of detection elements to the extender, block 406. A wash step is then performed to remove the excess target solutions, enzymes, detection elements, and any other unwanted compounds, block 408. A detection operation can then be performed to analyze and identify the target, block 410.

The single solution applied in step 404 of FIG. 4 is formulated to meet one or more characteristics relating to encouraging the enzymatic reaction as well as the hybridization operation of the probe and target sequences. Certain well known and commonly used hybridization solutions are generally optimized for hybridization processes, but may discourage or even prevent enzymatic reactions. For example, a solution that contains formamide (formic acid) may denature the proteins in the enzyme solution.

In the two reaction process of FIG. 2, the binding of target to probe is an equilibrium reaction such that only some portion of the target is bound during hybridization, and there may be circumstances in which some target is washed off in the first wash step 208. In the single solution process of FIG. 4, all the target is available for hybridization and the enzymatic reaction, thus, if allowed to run long enough, all target will be bound. This increases the sensitivity of the hybridized probe.

The single solution is selected with is configured to satisfy certain requirements, such as ionic strength, pH, and the presence of protein stabilizers. The solution may be optimized for enzymatic reactions rather than for hybridization, or vice-versa. In one embodiment, the single solution is formulated as shown in Table 1:

TABLE 1

| | |
|---|---|
| BUFFERS/ | Buffer (Tris-HCl): 10 mM-20 mM |
| STABILIZERS | (PH. 7.9-8.8 at 25° C.) |
| | Monovalent ion (K⁺, Na+, or |
| | NH₄⁺): 10 mM-50 mM |
| | Divalent ion (Mg⁺⁺): 1-2.5 mM |
| | BSA: 1 mg/ml |
| | DTT (dithiothreitol): 1 mM |
| | Detergent (Triton X-100): 0.1% |
| DETECTION | Cy⁵⁻dATP, Cy³-dATP (DYE) or p³²-dATP: |
| ELEMENTS | 0.1 uM to 1 uM (RADIOACTIVE) or |
| | Biotin-dATP (BIOLOGICAL) |
| ENZYME | Klenow: 5 units to 50 units or other enzyme |
| ADDED REAGENTS | Betaine (1 M), and/or DMSO (5-10%) and/or, |
| (optional) | Glycerol (at 5-10%) |

The constituent components for the above composition are given in relative concentrations (moles/liter). In general, the buffer/stabilizer components include a salt solution (e.g., Tris-Hydrochloride) that buffers the pH and stabilize the nucleic acids, and various other components including a carrier protein (e.g., bovine serum albumin, BSA), monovalent ion, divalent ion, dithiothreitol (DTT), and a detergent (e.g., Triton-X). The enzymes used for the single solution process of FIG. 4 and Table 1 (as well as the two-solution process of FIG. 2) may be any appropriate enzyme depending on the application. Such enzymes include Klenow, exo-Klenow, BSTL1 (BST DNA Polymerase Large Fragment), VENT polymerase, Therminator II DNA polymerase, or any other appropriate enzyme. The enzyme unit (e.g., 5-50 units) represents the enzymatic activity level required to convert 10 nM of dNTP's to an acid insoluble material in 30 minutes at 37° C., for the case of Klenow. Other enzymes may require different temperatures. For example, for BSTL1, the temperature is 65° C., and for VENT, the temperature is 75° C. The added reagents add further stabilization. For example, glycerol and dimethyl sulfoxide (DMSO) increase the stability of the enzyme, and betaine normalizes the base pair (AT or CG) binding. The added reagents are optional and can be used in appropriate combinations or amounts depending upon the application.

The solution composition of Table 1 is an example of one possible composition for a single solution for performing both hybridization and an enzymatic reaction to bind detection elements to an extender in one-step, under an embodiment. Other compositions may be formulated by substituting the various constituents with equivalent or similar compounds. In general, the one-step composition of Table 1 should include the buffers and stabilizers listed, at least one detection element and at least one enzyme, such as Klenow. The added reagents are not strictly necessary, and may be added as needed for stabilization or other purposes. In one embodiment, the solution may be provided in pre-mixed or partially pre-mixed form, or it may be provided as a kit with the constituent elements provided for mixing and application at a particular site. In general, no specific requirements are necessary for mixing procedures, other than normal biological laboratory procedures regarding operating conditions, ambient temperatures, cleanliness, and so on. In an embodiment, the solution of Table 1 is mixed with an amount of target solution containing one or more nucleic acids. This final mixture can then be applied to the probe microarray slide.

For the solution of Table 1, the minimum input amount of RNA is on the order of 0.5 μg total RNA, and can be as low as 0.1 to 0.2 μg total RNA. No input purification is required to remove large RNAs, and even degraded RNA can be used with no risk of non-specific signals and degradation of detection, since only intact RNA will be extended through the enzymatic reaction. The hybridization temperature can be in the range of 25° C.-75° C., or even up to 80° C. if VENT polymerase is used. The relaxed temperature requirements allows for the adjustment of specificity at will, and an overall higher degree of specificity. The process equalizes the stability of different probes by adding approximately 20 nucleotides through the extender structure, allowing much more uniform results. In this manner, weak signals will be stabilized and rescued. The one-step process generally avoids loss of target even at high temperature

In a typical application, the single solution process allows the hybridization step to occur much more quickly than in conventional methods. A typical hybridization step may take on the order of 8 to 20 hours to ensure that as many target sequences as possible are bound to the probe sequences. In the single solution process, the enzymatic reaction facilitates or, in effect, accelerates the hybridization process so that it occurs much more quickly, such as on the order of two hours or less, as opposed to 16 hours. The process effectively ends when all of the target within the solution is bound to the probe, thus, virtually no target is left over as excess. The single solution (or one-step) process also simplifies the overall hybridization process by requiring only a single wash step, which may be very stringent without risk of causing a loss of target. This due to the increased stability and increased length (up to 43mer) of the overall probe comprising the target and the complementary extender,

Unlike present known hybridization processes, the temperature of the hybridization process for FIG. 4 need not be optimized for hybridization. For example, the process can run at a higher temperature. For example, present hybridization processes on a 23mer sequence can occur at 37 degrees Celsius and up to 42 degrees Celsius. The one-step process can now be performed at 65 degrees Celsius for hybridization for a 23mer.

The use of the single solution hybridization method, alleviates many of the drawbacks associated with present diffusion-based hybridization schemes. The principal disadvantage of present hybridization methods is the required tradeoff between specificity and sensitivity. In general, any increase in sensitivity reduces the specificity of the hybridization. In the one-step hybridization process that utilizes a single enzymatic and target solution, an increase in the sensitivity of hybridization does not necessarily reduce the specificity due to the fact that the enzymatic reaction drives the hybridization reaction. This decoupling of these important parameters greatly improves detectability, while reducing the time required for the overall process, as well as the amount of target solution required.

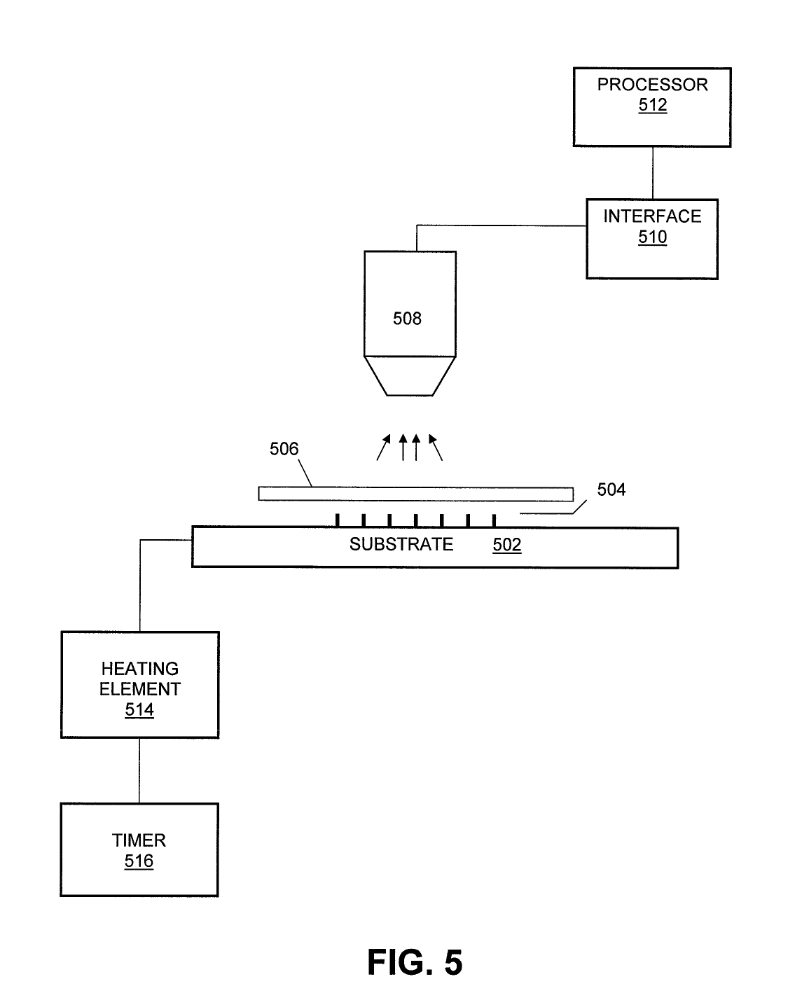

Embodiments of the microRNA detection method utilizing an enzymatic solution in either a two-part solution with separate hybridization and enzymatic reactions, or a one-part solution with concurrent hybridization and enzymatic reactions is intended for use in conjunction with a nucleic acid detection system. FIG. 5 illustrates a nucleic acid detection system for use with a microarray hybridized through an enzymatic reaction, under an embodiment.

The microarray chip of FIG. 5 generally comprises a solid substrate 502 on which a probe or series of probes 504 are immobilized. The probe 504 can be immobilized on an untreated or treated surface of the substrate through covalent bond for specific detection of a complementary interaction with a target sequence. The target sequence is applied through a target solution introduced onto the surface of the substrate 502. A slide 506 or other enclosing structure can be placed over the probe area 504. This area may be a amplification and hybridization chamber that may be hermetically sealed or open on any of the sides. The substrate may be thermoconductive and coupled to or exposed to a temperature control or heating source 514 that provides a controllable temperature for the hybridization and enzymatic reactions. The heating source may be controlled through any appropriate means, including a timer 516.

A scanner 508 detects the presence of the hybridized probe sequences using an appropriate receiver for the detection elements. For example, if the detection elements are fluorescent molecules, scanner 508 is a fluorescence scanner for detection of the fluorescent hybridization signals. The output of the scanner is provided through interface 510 to a processor 512 for analysis of the detection signals. The processor may execute one or more programs that analyze and assess the detected target nucleotide sequences. Assessing refers to the quantitative and/or qualitative determination of the hybrid formed between the probe and nucleotide sequence. This can be an absolute value for the amount or concentration of the hybrid, or an index or ratio of a value indicative of the level of the hybrid.

The microarray detection system of FIG. 5 can be used to assay a large number of nucleic acids simultaneously and gene expression patterns under a given condition can be rapidly analyzed. The system can be used in conjunction with a gene chip or biochip system comprising an array of oligonucleotides or nucleic acids immobilized on the substrate surface. Such a microarray can be used for any suitable purpose, such as screening an RNA sample, single nucleotide polymorphism, detection, mutation analysis, disease or infection prognosis, genome comparison, and other like applications.

As used herein, the term “nucleic acid” refers to multiple linked nucleotides (i.e., molecules comprising a sugar lined to an exchangeable organic base, which is either a pyramidine (Cytosine(C), thymidine (T), or uracil (U)), or a purine (e.g., adenine(A), guanine (G)). A nucleic acid also refers to oligoribonucleotides as well as oligodeoxyribonucleotides, as well as polynucleosides and any other organic base containing nucleic acid. The organic bases include adenine, uracil, guanine, thymine, cytosine and inosine. The nucleic acids may be single-stranded or double-stranded, and may be obtained from natural sources or through a synthetic process.

Embodiments of the microarray system as described and illustrated may be implemented in or used in conjunction with microarray-based, bio-information collection system, including an RF reader/writer, utilizing a computer, or computers executing software instructions. The computer may be a standalone computer or it may be networked in a client-server arrangement or similar distributed computer network. For the purposes of the present description, the term “processor” or “CPU” (Central Processing Unit) refers to any machine that is capable of executing a sequence of instructions and should be taken to include, but not be limited to, general purpose microprocessors, special purpose microprocessors, Application Specific Integrated Circuits (ASICs), multi-media controllers, digital signal processors, and micro-controllers, etc.

A memory device or devices may be associated with the system illustrated in FIG. 5, and may be embodied in a variety of different types of memory devices adapted to store digital information, such as static random access memory (SRAM), dynamic random access memory (DRAM), synchronous dynamic random access memory (SDRAM), and/or double data rate (DDR) SDRAM or DRAM, as well as standard non-volatile memory such as read-only memory (ROM). Moreover, the memory device(s) may be embodied in off-chip variations through a memory interface that allows the transfer of data to other storage devices such as hard disk drives, floppy disk drives, flash drives, optical disk drives, etc., and appropriate interfaces.

While the term “component” is generally used herein, it is understood that “component” includes circuitry, components, modules, and/or any combination of circuitry, components, and/or modules as the terms are known in the art.

Embodiments are directed to a method for detecting a nucleic acid on a microarray surface, comprising: providing a probe having a homogenous sequence extender and a sequence of nucleotide bases that is complementary to a specific nucleic acid sequence of interest; hybridizing the probe nucleotide sequences with a target solution containing the sequence of interest; and adding an enzymatic solution comprising detection elements and enzymes to the hybridized probe to actuate an enzymatic reaction on the microarray surface that binds the detection elements to the extender to facilitate detection The method can further comprise preparing the target solution and enzymatic solution as a single solution that includes the target, the detection elements, and the enzymes, and wherein the step of hybridizing the probe nucleotide sequences occurs concurrently with the binding of the detection elements to the extender.

Embodiments are further directed to a method for detecting a nucleic acid on a microarray surface, comprising: providing a probe having an extender and a sequence of nucleotide bases that is complementary to a specific nucleic acid sequence of interest; adding a single solution comprising a target containing the sequence of interest, a quantity of detection elements and a quantity of enzymes to the hybridized probe; hybridizing the probe nucleotide sequences with the target; and performing an enzymatic reaction on the microarray surface to bind the detection elements to the extender to facilitate detection.

With regard to a system or apparatus, embodiments are directed to a microarray detection system comprising: a substrate containing one or more immobilized probe sequences on the substrate surface, at least some of the probe sequences hybridized to target sequences with detection elements bound to the probe sequences through an enzymatic reaction performed on the substrate surface; a detector configured to detect the detection elements; one or more environmental controls configured to control a hybridization reaction creating the hybridized target sequences; and a processor coupled to the detector and configured to analyze the detected detection elements to assay the target sequence. In this detection system, the hybridization reaction may be performed by applying a target solution containing the target sequences to the substrate surface, and the enzymatic reaction is performed by applying a separate enzymatic solution to the substrate surface, the enzymatic solution comprising an enzyme and the detection elements. Alternatively, the hybridization reaction is performed concurrently with the enzymatic reaction by applying a single solution containing the target sequences, an enzyme and the detection elements.

Embodiments are further directed to a solution for simultaneously hybridizing a probe sequence and binding a detector element to a portion of the probe sequence comprising: a buffer solution containing an amount of salt solution (e.g., Tris-hydrochloride) mixed with an amount of monovalent ion, an amount of divalent ion, an amount of carrier protein (e.g., bovine serum albumin, BSA), an amount of dithiorthreitol, and an amount of detergent; an enzymatic component comprising an amount of enzyme; and a detection element component comprising an amount of markers to be linked to the portion of the probe sequence; and which are all mixed with a target solution of nucleic acids. The solution may further comprise a reagent component comprising respective amounts of one or more stabilizing elements selected from the group consisting of: betaine, dimethyl sulfoxide (DMSO), and glycerol. The solution may be provided in kit form for application as a single enzymatic solution to the probe sequence, and wherein the solution acts to build a complementary extender sequence bound to the probe sequence through an enzymatic reaction on a substantially homogenous extender portion of the probe sequence. In an embodiment, the single enzymatic solution operates to simultaneously cause hybridization of a portion of the probe sequence with one or more targets in the target solution, and bind the detection element to the extender portion of the probe sequence.

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number, respectively. Additionally, the words “herein,” “hereunder,” “above,” “below,” and words of similar import, when used in this application, refer to this application as a whole and not to any particular portions of this application. When the word “or” is used in reference to a list of two or more items, that word covers all of the following interpretations of the word: any of the items in the list, all of the items in the list, and any combination of the items in the list.

The above description of illustrated embodiments of the microarray system is not intended to be exhaustive or to limit the invention to the precise form disclosed. While specific embodiments of, and examples for, the invention are described herein for illustrative purposes, various equivalent modifications are possible within the scope of the invention, as those skilled in the relevant art will recognize. The various arrangements and operations described may be performed in a very wide variety of different microarray architectures, and although specific configurations are described herein, none are intended to be limiting or exclusive.

In general, in the following claims, the terms used should not be construed to limit the system and method to the specific embodiments disclosed in the specification and the claims, but should be construed to include any arrangements and methods that operate under the claims. Accordingly, the apparatus and method is not limited by the disclosure, but instead the scope is to be determined entirely by the claims.

While certain aspects of the system and method are presented below in certain claim forms, the inventors contemplate the various aspects of the system and method in any number of claim forms. Accordingly, the inventors reserve the right to add additional claims after filing the application to pursue such additional claim forms for other aspects of the described embodiments.

What is claimed is:

1. A method for detecting a nucleic acid on a microarray surface, comprising:
providing a probe having a homogenous sequence extender and a sequence of nucleotide bases that is complementary to a specific nucleic acid sequence of interest;
hybridizing the probe nucleotide sequences with a target solution containing the sequence of interest; and
adding an enzymatic solution comprising detection elements and enzymes to the hybridized probe to actuate an enzymatic reaction on the microarray surface that binds the detection elements to the extender to facilitate detection.

2. The method of claim 1 further comprising preparing the target solution and enzymatic solution as a single solution that includes the target, the detection elements, and the enzymes, and wherein the step of hybridizing the probe nucleotide sequences occurs concurrently with the binding of the detection elements to the extender.

3. The method of claim 1 wherein the substrate is selected from the group consisting of: glass, plastic, metal, silicon, cellulose, and polymer membranes.

4. The method of claim 3 further comprising coating the surface with a protein-based substance to facilitate an enzymatic reaction by stabilizing a protein structure near the surface.

5. The method of claim 4 further comprising applying reagents including bovine serum albumin (BSA) to the surface to increase the stability of protein structures to facilitate the enzymatic reaction.

6. The method of claim 1 wherein the enzyme is selected from the group consisting of: Klenow, exo-Klenow, Therminator II DNA polymerase, VENT polymerase, and BSTL1.

7. The method of claim 1 wherein the specific nucleic acid sequence of interest comprises an miRNA sequence, and wherein the extender comprises one of a polyT sequence, a polyA sequence, a polyC sequence, and a polyG sequence.

8. The method of claim 7 further comprising:
hybridizing a quantity of the probe nucleotide sequences at a defined temperature for a defined time;
washing the hybridized quantity at high stringency;
incubating the hybridized quantity with Klenow and Biotin-conjugated nucleotides;
binding the hybridized quantity with an Avidin-conjugated detection element.

9. The method of claim 1 further comprising:
performing a first wash after the hybridization step to remove non-specific sequences;
performing a second wash after the step of adding the solution to remove the excess detection elements and enzymes; and
performing a detection operation to detect the detection elements.

10. The method of claim 1 wherein the detection elements are selected from the group consisting of: fluorescent elements, biological elements, and radioactive elements.

11. A method for detecting a nucleic acid on a microarray surface, comprising:
providing a probe having an extender and a sequence of nucleotide bases that is complementary to a specific nucleic acid sequence of interest;
adding a single solution comprising a target containing the sequence of interest, a quantity of detection elements and a quantity of enzymes to the hybridized probe;
hybridizing the probe nucleotide sequences with the target;
performing an enzymatic reaction on the microarray surface to bind the detection elements to the extender to facilitate detection.

12. The method of claim 11 further comprising washing the hybridized probe to remove non-specific sequences, excess detection elements, and enzymes.

13. The method of claim 11 wherein the enzyme is selected from the group consisting of: Klenow, exo-Klenow, Therminator II DNA polymerase, VENT polymerase and BSTL1.

14. The method of claim 11 wherein the substrate is selected from the group consisting of: glass, plastic, metal, and silicon.

15. The method of claim 14 further comprising coating the surface with a protein-based substance to facilitate an enzymatic reaction by stabilizing a protein structure near the surface.

16. A method comprising:
providing a probe nucleotide sequence;
providing an extender structure comprising a plurality of nucleotides coupling the probe nucleotide sequence to a substrate surface;
hybridizing a target to the probe nucleotide sequence to produce a hybridized probe sequence; and
applying a single enzymatic solution to the hybridized probe sequence to build a complementary extender sequence bound to the hybridized probe sequence through an enzymatic reaction on the hybridized probe sequence, the enzymatic solution including detection elements detectable through a detection process and bound to the extender structure

17. The method of claim 16 wherein the step of hybridizing the target to the probe comprises applying a target solution containing a target sequence that is complementary to the probe sequence.

18. The method of claim 17 further comprising detecting the detectable elements bound to the extender structure to identify and assess the target sequence.

19. The method of claim 16 wherein the enzymatic reaction causes the detection elements to be bound to the extender structure.

20. The method of claim 19 wherein the detection elements are selected from the group consisting of: fluorescent elements, biological elements, and radioactive elements.

21. The method of claim 16 wherein the single enzymatic solution comprises stabilizer components, detection element components, enzyme, and added reagents.

22. The method of claim 21 wherein the detection elements are selected from the group consisting of: fluorescent elements, biological elements, and radioactive elements, and the enzyme is selected from the group consisting of: Klenow, exo-Klenow, Therminator II DNA polymerase, VENT polymerase, and BSTL1, and further wherein the stabilizer components include a salt composition and a one or more ion compositions.

23. A microarray detection system comprising:
a substrate containing one or more immobilized probe sequences on the substrate surface, at least some of the probe sequences hybridized to target sequences with detection elements bound to the probe sequences through an enzymatic reaction performed on the substrate surface;
a detector configured to detect the detection elements;
one or more environmental controls configured to control a hybridization reaction creating the hybridized target sequences; and
a processor coupled to the detector and configured to analyze the detected detection elements to assay the target sequence.

24. The detection system of claim 23 wherein the hybridization reaction is performed by applying a target solution containing the target sequences to the substrate surface, and the enzymatic reaction is performed by applying a separate enzymatic solution to the substrate surface, the enzymatic solution comprising an enzyme and the detection elements.

25. The detection system of claim 23 wherein the hybridization reaction is performed concurrently with the enzymatic reaction by applying a single solution containing the target sequences, an enzyme and the detection elements.

26. The detection system of claim 23 wherein the detection elements are selected from the group consisting of: fluorescent elements, biological elements, and radioactive elements.

27. The detection system of claim 26 wherein the enzyme is selected from the group consisting of: Klenow, exo-Klenow, Therminator II DNA polymerase, VENT polymerase, and BSTL1.

28. The detection system of claim 23, wherein the substrate is selected from the group consisting of glass, plastic, silicon, cellulose, and polymer membranes.

29. The detection system of claim 28, wherein the shape of the substrate is selected from the group consisting of a rectangle, square, circle, triangle, and polygon.

30. A solution for simultaneously hybridizing a probe sequence and binding a detector element to a portion of the probe sequence comprising:
a buffer solution containing an amount of a salt solution mixed with an amount of monovalent ion, an amount of divalent ion, an amount of a carrier protein, an amount of dithiorthreitol, and an amount of detergent;
an enzymatic component comprising an amount of enzyme; and
a detection element component comprising an amount of markers to be linked to the portion of the probe sequence; and
a target solution of nucleic acids.

31. The solution of claim 30 further comprising a reagent component comprising respective amounts of one or more stabilizing elements selected from the group consisting of: betaine, dimethyl sulfoxide (DMSO), and glycerol.

32. The solution of 30 wherein the enzyme is selected from the group consisting of: Klenow, exo-Klenow, Therminator II DNA polymerase, VENT polymerase, and BSTL1; and wherein the carrier protein comprises bovine serum albumin (BSA), and further wherein the salt solution comprises Tris-hydrochloride.

33. The solution of claim 32 wherein the detection elements are selected from the group consisting of: fluorescent elements, biological elements, and radioactive elements.

34. The solution of claim 33 wherein the solution components are provided in kit form for application as a single enzymatic solution to the probe sequence, and wherein the solution acts to build a complementary extender sequence bound to the probe sequence through an enzymatic reaction on a substantially homogenous extender portion of the probe sequence.

35. The solution of claim 34 wherein the single enzymatic solution operates to simultaneously cause hybridization of a portion of the probe sequence with one or more targets in the target solution, and bind the detection element to the extender portion of the probe sequence.
